(19) United States
(12) Patent Application Publication
(10) Pub. No.: US 2019/0175271 A1
(43) Pub. Date: Jun. 13, 2019

(54) MICROWAVE INSTRUMENT

(71) Applicant: Emblation Limited, Alloa (GB)

(72) Inventors: Gary Beale, Dunblane (GB); Eamon McErlean, Alloa (GB); Matthew Donal Kidd, Stirling (GB)

(73) Assignee: Emblation Limited, Alloa (GB)

(21) Appl. No.: 16/326,834

(22) PCT Filed: Aug. 25, 2017
(86) PCT No.: PCT/GB2017/052499

§ 371 Date: Feb. 20, 2019

(30) Foreign Application Priority Data
Aug. 26, 2016 (GB) 1614581.5

(51) Int. Cl. A61B 18/18; A61N 5/04

(52) U.S. Cl. CPC A61B 18/1815; A61N 5/025; A61B 2018/1861; A61N 5/045

(57) ABSTRACT

A microwave system comprises a microwave generator and a microwave cable apparatus comprising a coaxial cable having a diagonally-angled end surface, wherein the microwave generator is configured to provide microwave energy to the cable apparatus at a frequency that provides directional radiation of microwave energy having a desired directionality from the diagonally-angled end surface.

### FIELD

The present invention relates to a microwave instrument, for example a directional microwave ablation instrument for the ablation of targeted tissue volumes.

### BACKGROUND

It is known to use microwaves for ablation of tissue. In some known microwave ablation systems, microwave energy is delivered to a radiating applicator that transfers the microwave energy into the tissue. A radiating element of the radiating applicator may be surrounded by tissue or placed in contact with tissue.

Microwave ablation may ablate tissue using a different mechanism to radio frequency (RF) ablation. In microwave ablation, tissue may be heated directly by a radiating antenna. In RF ablation, heat may be created by resistive heating caused by an electric current passing through the tissue. RF heating may require an electrically conductive path. RF ablation may use frequencies in the range of, for example, 350 to 500 kHz.

An RF ablation device may affect areas in the path between conductors. The area affected by a microwave ablation device may in some circumstances be limited only by tissue penetration properties. A mechanism of microwave ablation is described in Lubner M G, Brace C L, Hinshaw J L, Lee F T Jr, Microwave tumor ablation: mechanism of action, clinical results, and devices, J Vasc Intery Radiol. 2010 August; 21(8 Suppl):S192-203.

Compared to other technologies, the use of microwaves in an ablation system may in some circumstances produce faster heating over a larger volume of tissue with less susceptibility to heat-sink effects. Microwave ablation may be effective in tissues with high impedance such as lung tissue or charred, desiccated tissue. Microwave energy may be capable of generating temperatures in excess of 100° C. Some microwave devices may not require grounding pads or other ancillary components.

In some microwave ablation systems, the radiating element is a simple coaxial monopole antenna having an isotropic radiation pattern. An operating frequency of the radiating element may be dictated by a length of the radiating monopole element and/or dielectric properties of a medium that the radiating element radiates into. The radiating monopole may be surrounded by a high-dielectric interface material. The high-dielectric interface material may control the frequency of operation and/or the efficiency of the radiator. One example of a monopole antenna is U.S. Pat. No. 6,823,218, in which a monopole antenna possessing isotropic radiation characteristics is described.

In some applications an applicator possessing a directional radiation pattern may be used. Delivery of energy may be targeted to specific areas, for example tumours, whilst avoiding other areas, for example, arteries, veins, nerves, or other important physiological features. Some applicators are known in which a reflector is used to provide directional radiation characteristics. An example of an antenna using a reflector may be U.S. Pat. No. 6,741,696. Further applicators are described in U.S. Pat. Nos. 7,301,131, 7,292,893, and U.S. Ser. No. 13/477,320.

An alternative to adding a reflector to the antenna may be to expose the central core of a coaxial transmission line (see, for example, U.S. Pat. Nos. 4,204,549 and 7,410,485). The length of the part of an inner conductor that is exposed (which may be referred to as a gap of exposed conductor) may be of a length equal to one quarter, or one half, of an effective wavelength. The length being one quarter or one half of a wavelength may promote a resonant effect with the coupled media. In EP 2,742,893, a central core in a customised transmission cable is exposed. The conductor is continuously tapered.

U.S. Pat. No. 7,180,307 describes a non-directional antenna in which a part of an outer conductor is removed and a corresponding part of an inner conductor is exposed, forming a 3D cone structure.

### SUMMARY

In a first aspect of the invention, there is provided a microwave system comprising: a microwave generator; and a microwave cable apparatus comprising a coaxial cable having a diagonally-angled end surface; wherein the microwave generator is configured to provide microwave energy to the cable apparatus at a frequency that provides directional radiation of microwave energy having a desired directionality from the diagonally-angled end surface.

The system may be configured to perform microwave ablation of tissue. The directionally-radiated microwave energy may be used to ablate tissue. A portion of the cable apparatus comprising the diagonally-angled end surface may act as an antenna. The cable apparatus may be simple and/or easy to manufacture. The system may provide directional radiation without the need for a choke and/or other components. By providing a simple system, reflection effects may be reduced or eliminated. Surface currents may be reduced or eliminated. Efficient transmission of radiation may be achieved.

The system may be used to deliver directional radiation to specific, controlled regions of tissue. By delivering directional rather than omnidirectional radiation, better targeting of radiation may be achieved.

The diagonally-angled end surface may form a diagonal angle with a longitudinal axis of the coaxial cable. The coaxial cable may comprise an inner conductor and an outer conductor. One side of the outer conductor may be longer than the inner conductor. An opposite side of the outer conductor may be shorter than the inner conductor. The end surface may comprise an end surface of the outer conductor and an end surface of the inner conductor.

A portion of the inner conductor may be exposed. The exposed portion of the inner conductor may be of any length. For example, the exposed portion of the inner conductor may be of a length that is not a quarter-wavelength or half-wavelength at a frequency of operation.

The coaxial cable may further comprise a dielectric interposed between the inner conductor and the outer conductor. The end surface may comprise an end surface of the dielectric.

The end surface may be substantially planar. A planar end surface may be simple to form.

The end surface may be substantially elliptical. The end surface may be curved. The end surface may be faceted.

The system may further comprise a controller configured to select the frequency of the microwave energy provided to the cable apparatus and/or a power of the microwave energy provided to the cable apparatus. The frequency may be selected in dependence on at least one of a reflection coefficient of the cable apparatus, a property of tissue to be treated, a volume of tissue to be treated, a type of treatment.

The desired directionality may comprise at least one of a desired depth of penetration into tissue, a desired radiation pattern, a desired linearity, a desired profile of radiated volume. By providing the desired directionality, specific regions of tissue may be addressed. The directionality may allow surgery to be performed in difficult area.

The coaxial cable may be flexible. The coaxial cable may be semi-rigid. The coaxial cable may be rigid.

The system may further comprise a catheter or trocar into which the cable apparatus is insertable. The catheter or trocar may be used to deliver the diagonally-angled end surface of the coaxial cable to tissue into which the microwave energy is to be radiated.

The cable apparatus may further comprise a coating that coats at least part of the end surface. The coating may be biocompatible. The coating may prevent short circuiting. The coating may reduce friction.

The cable apparatus may further comprise a jacket surrounding the outer conductor. The diagonal angle of the diagonally-angled end surface may be between 10° to 85°. The diagonal angle may be between 10° and 30°, between 30° and 60°, or between 60° and 85°. The diagonal angle may be less than 30°, less than 45°, or less than 60°. The diagonal angle may be greater than 45°, greater than 60°, or greater than 80°.

The diagonal angle of the diagonally-angled end surface may be selected in dependence on at least one of: a volume of tissue to be treated, a dielectric constant of tissue to be treated, a type of treatment.

The frequency may be between 900 MHz and 30 GHz. The frequency may be about 915 MHz, about 2.45 GHz, about 5.8 GHz, about 8.0 GHz, or about 24.125 GHz.

A diameter of the coaxial cable may be is between 0.1 mm and 25 mm.

The end surface may be alignable with a tissue feature, so as to radiate directionally into the tissue feature.

In a further aspect of the invention, there is provided a method of performing a tissue heating process comprising: generating microwave energy by a microwave generator; providing the microwave energy to a microwave cable apparatus comprising a coaxial cable having a diagonally-angled end surface; and heating tissue by directional radiation of microwave energy having a desired directionality from the diagonally-angled end surface.

The tissue heating may be so as to perform tissue ablation. The tissue heating may be so as to cause tissue hyperthermia.

In a further aspect of the invention, which may be provided independently, there is provided a microwave transmitting coaxial probe comprising an inner conductor, and outer conductor, and a dielectric interposed between the inner conductor and the outer conductor. A top portion of the coaxial probe may have a substantially planar shape across the inner conductor and the outer conductor so that a portion of the inner conductor and a portion of the dielectric are exposed. A height of the substantially planar shape portion may be set at a value at which a reflection coefficient exhibits substantially a minimum value. The inner conductor may be partially exposed from and partially covered by the dielectric. A biocompatible material may form an insulator between the cut face of exposed conductors, insulating elements, and tissue when in use.

There may also be provided an apparatus or method substantially as described herein with reference to the accompanying drawings.

Any feature in one aspect of the invention may be applied to other aspects of the invention, in any appropriate combination. For example, apparatus features may be applied to method features and vice versa.

### DETAILED DESCRIPTION OF EMBODIMENTS

Embodiments of the invention are now described, by way of non-limiting examples, and are illustrated in the following figures, in which:—

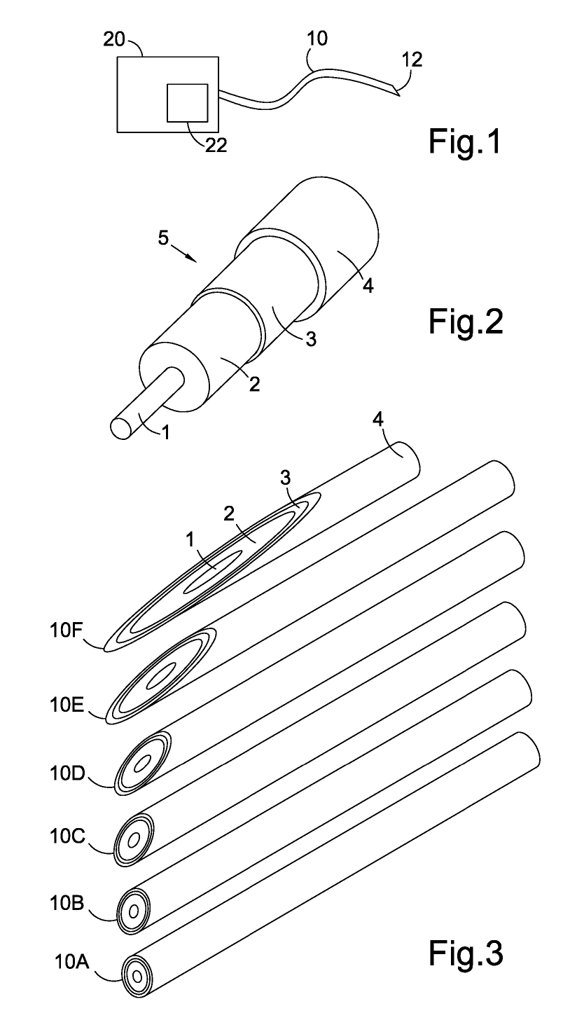

FIG. 1 is a schematic diagram a microwave system in accordance with an embodiment;

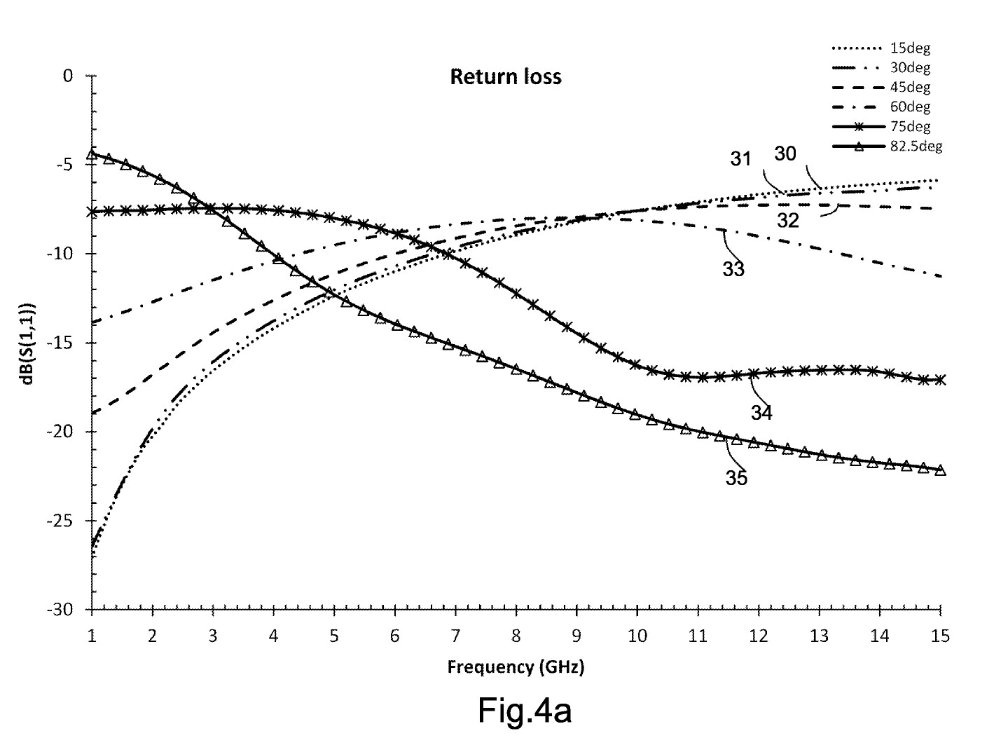

FIG. 2 is a schematic diagram showing an isometric view of constituent parts of a coaxial cable;

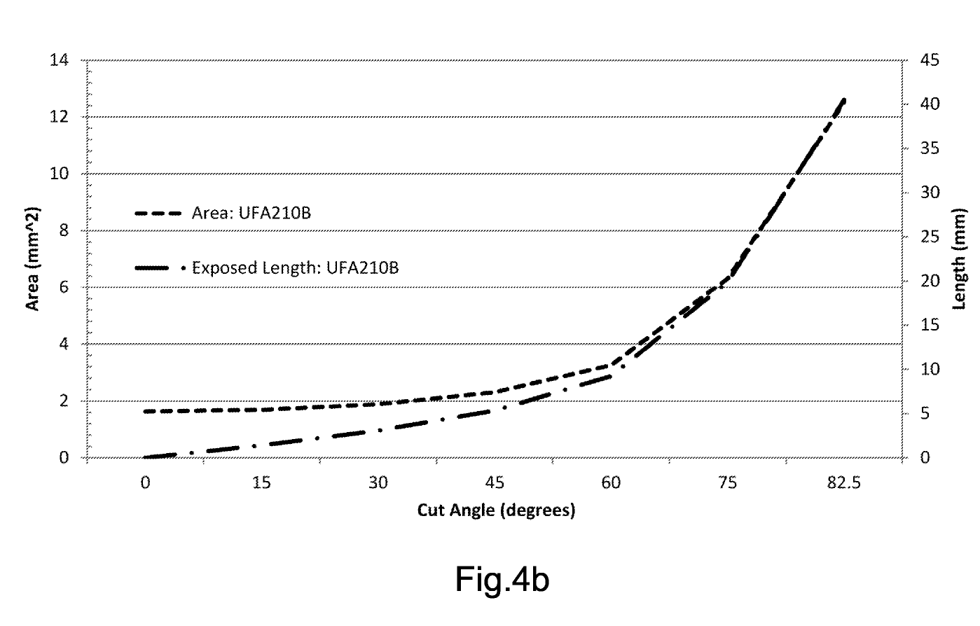

FIG. 3 is a schematic diagram showing isometric views of coaxial cables having cuts at 15°, 30°, 45°, 60°, 75° and 82.5° respectively;

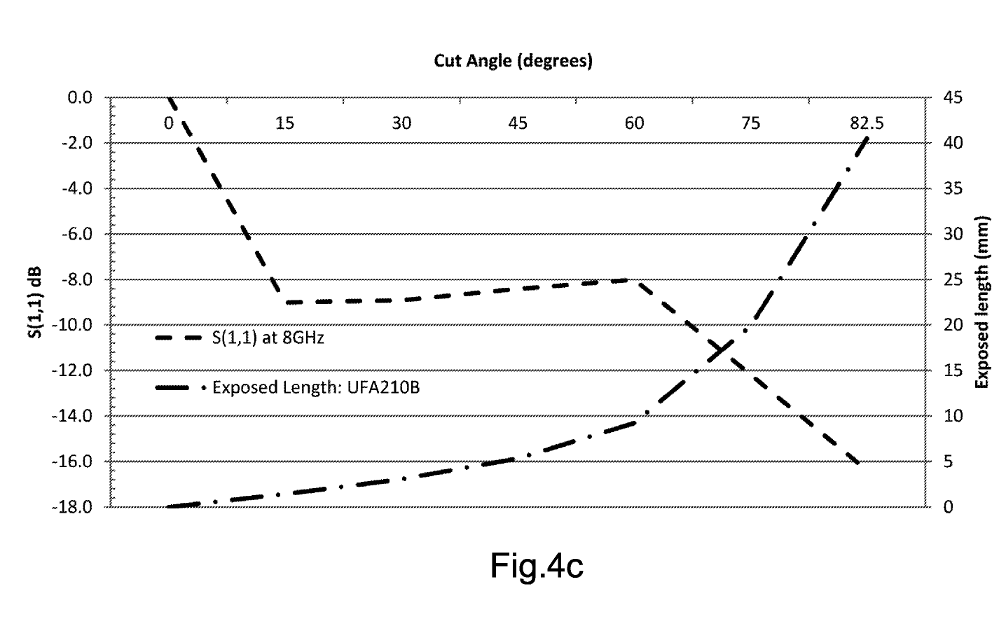

FIG. 4a is a plot showing attenuation responses for coaxial cables having cuts at 15°, 30°, 45°, 60°, 75° and 82.5° respectively;

FIG. 4b is a plot of area versus cut angle;

FIG. 4c is a plot of return loss versus cut angle;

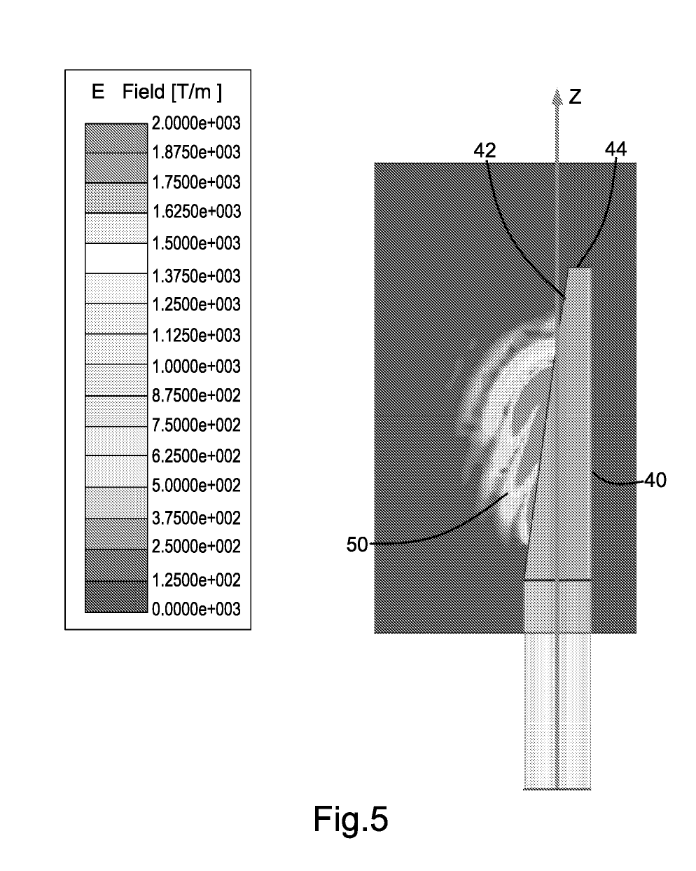

FIG. 5 is a plot of a modelled radiated field for an antenna of an embodiment;

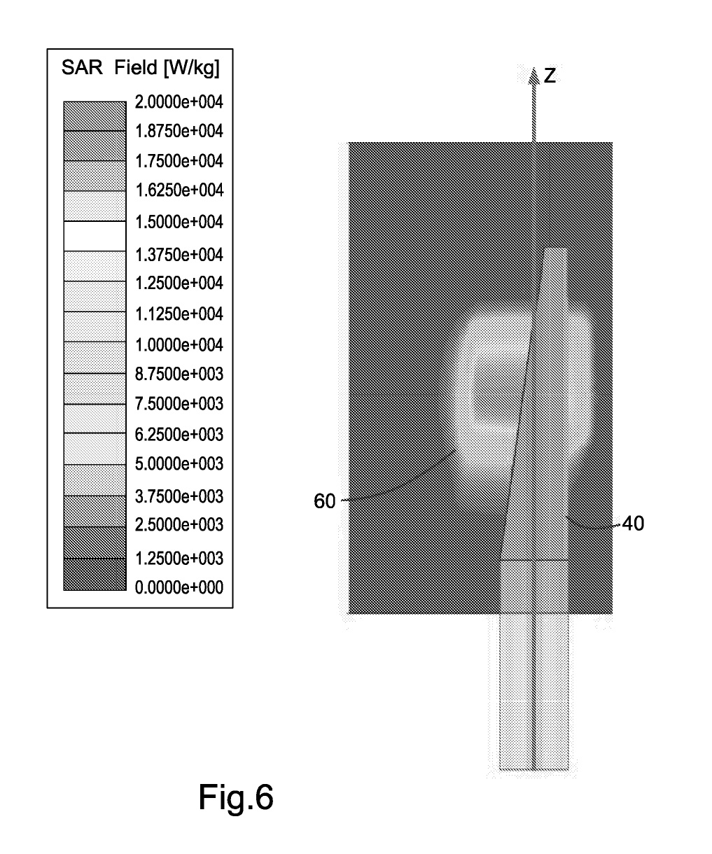

FIG. 6 is a plot of modelled specific absorption rate for an antenna of an embodiment;

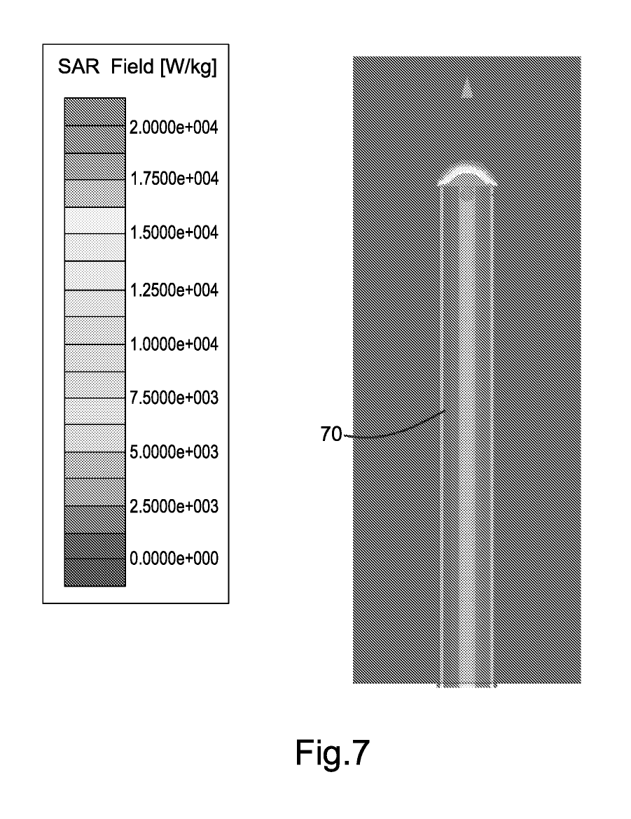

FIG. 7 is a plot of modelled specific absorption rate for an antenna having a 0° cut angle;

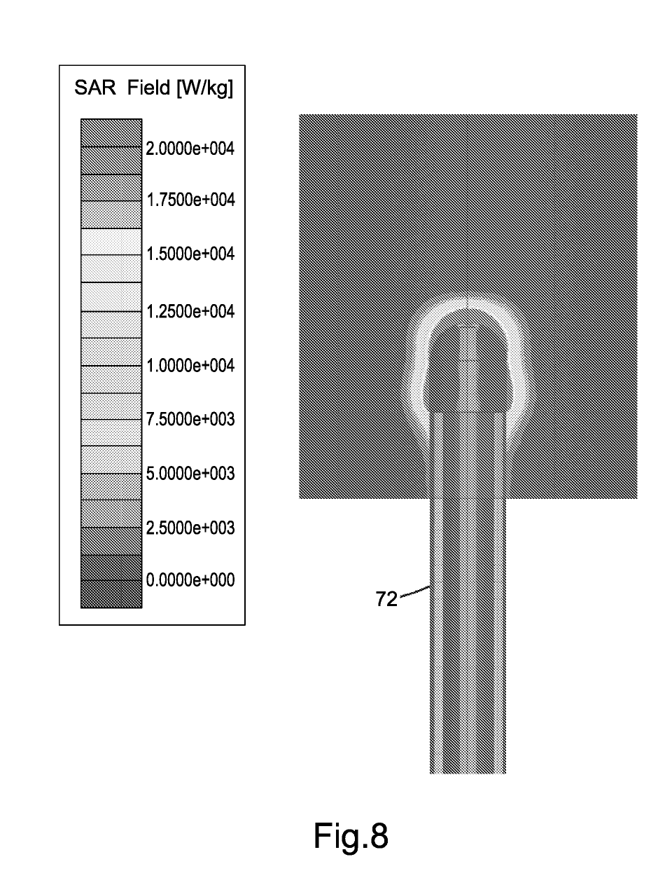

FIG. 8 is a plot of modelled specific absorption rate for an antenna;

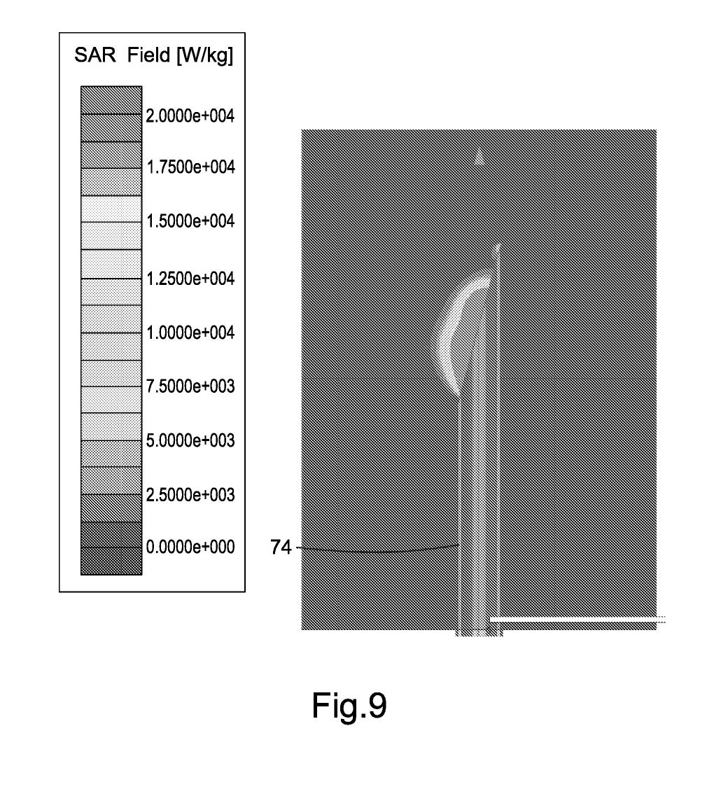

FIG. 9 is a plot of modelled specific absorption rate for an antenna of an embodiment;

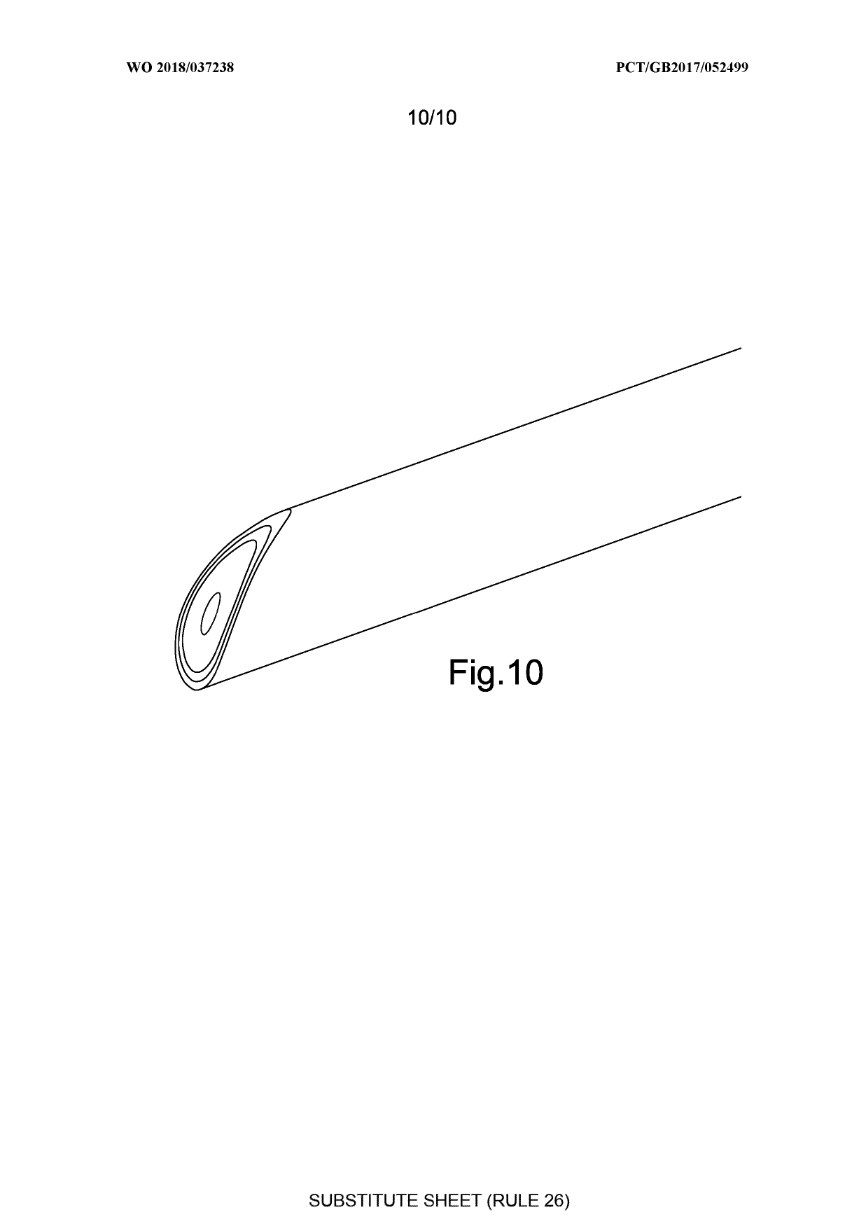

FIG. 10 is a schematic diagram showing an isometric view of a coaxial cable having a curved end surface.

FIG. 1 is a schematic diagram illustrating a microwave system according to an embodiment. The microwave system comprises a directional microwave ablation instrument for the targeted ablation of biological tissue. In further embodiments, a microwave system may be configured to perform any heating of tissue, which may or may not comprise ablation. For example, the heating of tissue may comprise hyperthermia.

The microwave system comprises a microwave generator 20 having a controller 22. A microwave cable apparatus 10 is coupled to the microwave generator 20. The microwave cable apparatus 10 comprises a coaxial cable. The end of the coaxial cable 10 that is not coupled to the microwave generator 20 has a diagonally-angled end surface 12 that has been formed by making a diagonal cut across the coaxial cable at an angle with respect to the longitudinal axis of the coaxial cable, thereby forming an antenna at the cut end of the coaxial cable. The antenna may also be called an emitter.

In the present embodiment, the cut across the cable is at an angle of 30° with respect to the longitudinal axis of the cable. In other embodiments, a different angle of cut may be used.

By exposing the inner conductor of the coaxial transmission line by means of an angled cut, a directional antenna is formed. If an appropriate frequency of microwave energy is applied to the directional antenna, the exposed planar surface 12 may radiate microwave energy.

FIG. 2 is representative of a flexible, semi-rigid or rigid coaxial cable construction. A coaxial cable 5 may comprise an inner conductor 1, insulator 2, outer conductor 3, and jacket 4. In some embodiments, the coaxial cable 5 may further comprise a cladding layer (not shown) between outer conductor 3 and jacket 4. In other embodiments, the coaxial 5 may comprise any suitable additional or alternative layer.

In the present embodiment the coaxial probe 10 of FIG. 1 comprises an SMA to BMA feed to a micro-coax (RTM) flexible cable (UFA210B). The inner conductor 1 comprises 19-strand silver plated copper. The insulator 2 comprises low density PTFE. The outer conductor 3 comprises an inner shield comprising silver plated copper foil and an outer shield comprising silver plated copper wire. The jacket 4 comprises fluorinated ethylene propylene (FEP).

The diameter of inner conductor 1 is 0.0565 inch (1.44 mm). The outer diameter of insulator 2 is 0.160 inch (4.06 mm). The outer diameter of the inner shield is 0.167 inch (4.28 mm). The outer diameter of the outer shield layer is 0.186 inch (4.72 mm). The outer diameter of the jacket 4 is 0.210±0.004 inch (5.33±0.1016 mm).

In other embodiments, a larger or smaller cable may be used. For example, a cable diameter having a diameter of 0.141 inch (3.58 mm) or 0.086 inch (2.18 mm) may be used. A size of the cable may be dependent on an application for which the cable is to be used. For example, a size of the cable may be selected based on a part of the body into which the cable is to be inserted.

A cross-sectional area of the inner conductor 1 may be approximately 10% of the cross-sectional area of the coaxial cable. A cross-sectional area of the insulator 2 may be approximately 65% of the cross-sectional area of the coaxial cable. A cross-sectional area of the outer conductor 3 may be approximately 25% of the cross-sectional area of the coaxial cable.

In other embodiments, different cables may be used. Any suitable coaxial cable may be used. The coaxial cable may be a rigid, semi-rigid or flexible cable.

Some examples of semi-rigid coaxial cables may comprise outer conductors of copper, aluminium, silver or stainless steel cladding or plating over a polytetrafluoroethylene (PTFE) insulator which carries a copper, aluminium or silver plated conductive centre conductor. The outer conductor may be covered with a stainless steel outer jacket to prevent corrosion or for biocompatibility reasons.

In some embodiments, the jacket 4 of the cable may be formed of any suitable jacket material. The jacket material may comprise at least one of stainless steel, polytetrafluoroethylene (PTFE), fluorinated ethylene propylene (FEP) and perfluoroalkoxy alkane (PFA). A cladding layer may surround the exterior of the outer conductor 3. The cladding layer may be formed of any suitable cladding material. The cladding material may comprise at least one of silver and stainless steel.

The outer conductor 3 may be formed of any suitable conductive material. The conductive material may comprise at least one of copper, aluminium and silver. The insulator 2 may be formed of any suitable dielectric insulator. The dielectric insulator may comprise PTFE. The insulator 2 may be a continuous integral dielectric material which extends along the length of the cable to the cut surface, as may be manufactured in common coaxial cable construction.

The inner conductor 1 may be formed of any suitable conductive material. The conductive material of the inner conductor 1 may comprise at least one of copper clad steel and silver coated copper alloys. The inner conductor 1 (which may also be called a central conductor) may be solid or may be made from multiple strands.

It has been found that when a planar diagonal cut is made across a coaxial cable and microwave energy is supplied to the coaxial cable, at some frequencies the microwave emission profile of the antenna formed by cutting the coaxial cable may become directional. In some circumstances the microwave emission profile may become highly directional. The microwave emission profile may become directional without the addition of any further mechanical features, for example without the addition of a reflector. With no or low reflection introduced (for example, with no or low reflection introduced by the introduction of additional mechanical features), efficient transfer of energy may be achieved across a significant operating bandwidth.

FIG. 3 shows a plurality of coaxial probes 10A to 10F, each comprising components 1, 2, 3, 4 as illustrated in FIG. 2. For each coaxial probe 10A to 10F, the inner (central) conductor 1 of the coaxial transmission line is exposed by means of a diagonally-angled cut. The cut follows a plane through all the features of the coaxial cable, comprising the inner conductor 1, the outer conductor 3, and the dielectric 2 interposed between the inner and outer conductors 1, 3. The resultant cut operation forms a planar portion of the inner conductor 1 and dielectric 2. The diagonal cut results in the planar portion being elliptical.

The centre conductor 1 is cut obliquely leading to partial exposure of the centre conductor 1 and insulation of the exposed centre conductor 1. The inner conductor is partially exposed from and partially covered by the dielectric 2.

The angles of cut for cables 10A, 10B, 100, 10D, 10E and 10F shown in FIG. 3 are 15°, 30°, 45°, 60°, 75° and 82.5° respectively. The angles of cut are described with reference to a cut perpendicular to the length of the cable, which would in this convention have an angle of cut of 0°.

For the cables shown in FIG. 3, the cut surface of the dielectric 2 is flush with the cut surfaces of the inner conductor 1 and outer conductor 3. In other embodiments, the cut surface of the dielectric 2 may be not flush with the cut surface of the inner conductor 1 and/or the cut surface of the dielectric 2 may be not flush with the cut surface of the outer conductor 3. In such embodiments, the antenna formed by the cut surface may still radiate. However, in some circumstances the antenna may be less directional than antennas in which the cut surface of dielectric 2 is flush with the cut surfaces of the inner conductor 1 and outer conductor 3.

In some circumstances, if the cut surface of dielectric 2 is not flush with the cut surfaces of the inner conductor 1 and outer conductor 3 there may be a risk of burning, as tissue trapped between an exposed conductor 1 and ground 3 may conduct some or most of the supplied energy.

In some circumstances, if there is retracted (less) dielectric, the antenna may have a poorer response, depending on what is in the resulting void (for example, air, tissue, or conductive material). The material in the void may impair the desired effect of radiating microwaves into a target tissue. In some circumstances, if there is protruding (more) dielectric, the efficiency of transmission may be reduced.

Multiple cut planes (such as those shown in FIG. 3) can form a range of features depending on the desired profile of radiated volume, for example as described below with reference to FIG. 4a.

The planar cut is made across the entire coaxial cable construction such that the central conductor 1 is elliptical in a plan view. The insulator 2, outer conductor 3 and jacket 4 may each be elliptical in plan view. Each coaxial probe 10A to 1° F. has a planar elliptical surface 12 at a diagonal angle to the length of the coaxial cable.

In some circumstances, the antenna may be easily fabricated from coaxial cable, for example from standard semi-rigid coaxial cable. The cable may be readily cut, milled or polished in any one of a number of ways to achieve a desired shape.

In the present embodiment, the planar cut is made with an accurate saw (mitre cutter). In other embodiments, the cable may be cut using any suitable method. For example, the cable may be cut by laser cutting or may be cut by a knife.

In the present embodiment, the end surface 12 is polished or lapped to a smooth finish. The polishing or lapping may ensure that the end surface 12 is planar. In other embodiments, any suitable finishing process may be used.

In the present embodiment, the end surface 12 is processed mechanically (polished or lapped) but no further component, for example no coating, is applied to the end surface 12. In another embodiment, the end surface 12 (which may be referred to as the resulting antenna face) may be covered by a protective biologically compatible coating made from an substantially electrically transparent material such as polytetrafluoroethylene (PTFE), polyetheretherketone (PEEK) or fluoroethylene polymer (FEP).

In various embodiments, a coating may be applied to the end surface 12. The coating may comprise a biocompatible material. The coating may form a biocompatible barrier layer. The coating may prevent the various materials used in the cable construction from contacting tissue when the antenna is in used. For example, the coating may prevent from contacting tissue any materials that would normally, if the cable had not been cut, have been protected by the outer jacket from contacting tissue. The coating may form an insulator between the cut face of exposed conductors and insulating elements and tissue when in use. The coating may comprise any suitable insulating material, for example PTFR, PEEK, FEB, or parylene. The coating may comprise a dielectric. The coating may stop short paths on tissue that might otherwise lead to a burn. The coating may reduce the chance of short circuiting in a conductive medium. The coating may act to aid insertion into tissue, for example by reducing friction. Friction against tissue may be reduced. Friction against other components may be reduced.

In use, the microwave generator 20 generates microwave energy. The microwave generator 20 supplies microwave energy to the coaxial probe 10, and at least some of the supplied microwave energy is radiated from end surface 12. The end surface 12 of the coaxial probe 10 is placed near to or contacting the tissue of a patient. Microwave energy is radiated by the end surface 12 into the tissue of the patient.

The microwave generator is configured to provide microwave energy to the end surface 12 at a frequency that provides directional radiation from the end surface 12. The frequency may be selected such that the directional radiation has a desired directionality, for example a desired radiation pattern.

The directionality of the radiation may result from the diagonally-angled end of the coaxial probe. In FIG. 3, it may be seen that for each coaxial probe, one side of the outer conductor being longer than the inner conductor, and an opposite side of the outer conductor being shorter than the inner conductor. Microwaves may not be able to propagate through the longer side of the conductor and thus the field may be biased to the exposed inner conductor and unshielded section. A direction of radiation may be normal to the cut surface. Radiation may be not symmetric about the longitudinal axis of the coaxial probe.

In the present embodiment, a frequency of the generated microwave energy is controlled by controller 22. Controller 22 also controls an amplitude of the generated microwave energy. Controller 22 may control the frequency and/or amplitude of the generated microwave energy in response to user input and/or in response to signals from one or more sensors (not shown). By changing the frequency and/or amplitude of the microwave energy, properties of a radiation field of the radiated microwave energy may be changed. A shape of a radiation field may be changed. A depth to which the radiation penetrates into tissue may be changed.

A frequency of the microwave energy may be between 900 MHz and 30 GHz. For example, the frequency may be 915 MHz, 2.45 GHz, 5.8 GHz, 8.0 GHz, or 24.125 GHz.

A frequency and/or power of the generated microwave energy may be selected in dependence on a property of tissue that is to be treated using the antenna, for example on the tissue dielectric constant. A frequency and/or power of the generated microwave energy may be selected based on the type of treatment to be performed (for example, ablation, or tissue heating that does not cause ablation). A frequency and/or power of the generated microwave energy may be selected based on a volume of tissue to be treated, for example based on the size or shape of a volume of tissue to be treated.

In some embodiments, the antenna is introduced into the body via a catheter or trocar. In such embodiments, a diameter of the coaxial cable may be such that the antenna can fit into the catheter or trocar used. For example, different catheter sizes may be used for catheters entering different parts of the body. A diameter of the coaxial cable may be appropriate to a diameter of a body part into which the coaxial cable is to be inserted by catheter. The catheter may deliver the antenna to a position adjacent to tissue within the patient, for example to the liver, heart, pancreas, or other organ.

In many embodiments, including embodiments in which the antenna is introduced via a catheter, there is no need for the antenna itself to penetrate the tissue, for example to pierce the skin of a subject. The antenna may be made in flexible materials and may not need to be load bearing. For example, the antenna may be formed from a coaxial cable that is capable of being bent. The antenna may be formed from a coaxial cable that would bend if pressure were applied to the tip of the antenna.

In some embodiments, a flat region formed by the end surface 12 is used as a directional surface applicator. The flat region, when used as a directional surface applicator, may be used to treat superficial skin or organ lesions or to coagulate along surfaces or edge faces. The flat region may be placed near to or contacting the tissue of a patient. The tissue may comprise external tissue (for example, skin) or internal tissue (for example, tissue that has been exposed during surgery).

The antenna may be larger or smaller depending on the application and delivery method.

Various embodiments have been simulated using a 3D simulation model. In this case, the simulation model is HFSS (Ansoft Corp) which is a Finite Element Method (FEM) based full wave electromagnetic solver.

Simulations may allow the calculation of a predicted response for coupling efficiency and specific absorption rate (SAR). SAR is a measure of the rate at which energy is absorbed by the human body when exposed to a radio frequency (RF) electromagnetic field.

FIG. 4a is a plot showing return loss against frequency for a set of angles of cut. An end surface may be described in terms of cut angle and/or in terms of a length of the cut, for example a dimension of the end surface 12.

Modelled return loss S₁₁ in decibels is plotted against frequency in GHz over a range from 0 GHz to 15 GHz.

The angles of cut modelling in the simulation for which results are shown in FIG. 4 are 15° (line 30), 30° (line 31), 45° (line 32), 60° (line 33), 75° (line 34) and 82.5° (line 35). The coaxial cable for which results are shown in FIG. 4a is the flexible coaxial cable of the present embodiment (UFA210B).

The choice of angle may match a desired size and shape of a treatment volume. The use of small angles may correspond to a shorter exposed conductor and may be used for a smaller volume. In FIG. 4a, the 75° and 82.5° angles decrease the return loss for part of the frequency range and so may improve the utility for transferring energy into a material.

Software modelling of the profile has shown that a shallow angle of cut with respect to a plane perpendicular to the length of the cable (e.g. shorter exposed conductor lengths) may initiate useful energy emission at a lower frequency than a larger angle of cut.

In some embodiments, a shorter exposed length of conductor may tend to work at a higher frequency.

In embodiments, the angle of the cut may be set at a value at which a reflection coefficient exhibits substantially a minimum value. A frequency may be selected to provide a desired directionality of radiation given the angle of cut.

In some embodiments, a dimension of the cut surface 12 may be chosen to be a half or quarter wavelength at a frequency of interest (which may be a half or quarter wavelength of that frequency in tissue).

In some circumstances, lower-frequency microwave radiation may penetrate further into tissue than higher-frequency microwave radiation. In some applications it may be desirable to penetrate further into the tissue than in other applications.

The directional microwave profile radiated at the end surface 12 may be controlled by the cut geometry. A coaxial cable having a particular cut geometry may be selected for a given application.

In some embodiments, a shallow (short) cut may be used to deliver deep, low frequency, ablation to a large area. In other embodiments, an acute (long) cut may be used to deliver precise, shallow, high frequency ablation.

A choice of cut angle may be derived from a desired coupling into specific properties. A choice of cut angle may be driven by the properties of a target tissue, for example the permittivity (dielectric constant) of the target tissue. For example, one tissue may require one angle while another tissue may require a different angle.

In determining which cut angle to use, one may look at an area versus return loss plot and then calculate an angle to produce an area yielding a desired return loss. Such plots are shown for a cable (UFA210B) in FIGS. 4b and 4c. FIG. 4b shows an example of a plot of area and length versus cut angle. FIG. 4c shows an example of a plot of return loss and exposed length versus cut angle, at 8 GHz.

A direction of the directional radiation may be not affected by frequency or by a material of the cable. An amount of energy transmitted may be affected by frequency and/or a material of the cable.

Given enough energy, any design may be able to ablate (e.g. to reach a desired temperature for ablation). However, how this is achieved may be a matter of efficiency. Angled cutting may improve efficiency by a varying amount. The amount to which efficiency is improved may depend on the geometry of the cable, the material of the cable and the frequency of the microwave energy.

A radiation field provided by the end surface 12 may be wider in one plane than in another plane. A radiation field provided by the end surface 12 may extend further along one axis than along another, perpendicular axis. For example, the radiation field may be wider in the direction of the long axis of the elliptical surface than in the direction of the short axis of the elliptical surface. A frequency and/or cut angle may be selected to provide a desired linearity of radiation.

Control of the directional microwave profile may allow for penetration in a specific zone that may be linear. A linear zone of penetration may suit, for example, coagulation requirements or linear lesion formation. The microwave profile may allow microwave penetration along the line of a linear feature (for example, a vessel) while limiting microwave penetration outside that linear feature. The antenna may be oriented so as to provide directional radiation that is substantially aligned with the linear feature.

FIG. 5 illustrates a radiated electric field 50 of an antenna 40 that is formed by cutting a coaxial cable using an angled cut to expose a cut surface 42. In the embodiment of FIG. 5, the angled cut is not made across the entire construction but instead intersects the end of the coaxial cable, such that a portion 44 of the end of the coaxial cable is substantially perpendicular to the longitudinal axis of the cable. The diagonally-angled end surface has the shape of a truncated ellipse.

FIG. 6 is a plot of the specific absorption rate of the antenna 40 of FIG. 5. The specific absorption rate is shown as a SAR field 60.

FIGS. 7, 8 and 9 are plots of the specific absorption rate of further antennas 70, 72, 74. FIG. 7 is a plot of the specific absorption rate of an antenna 70 having a cut angle of 0° (e.g. being formed by a straight rather than a diagonal cut). FIG. 8 is a plot of the specific absorption rate of an antenna 72 that may be referred to as a traditional ceramic addition, and shows no directional capability. The specific absorption rate is axi-symmetric in the case of antenna 72. FIG. 9 is a plot of the specific absorption rate of an antenna 74 having a cut angle of 75°. The specific absorption rate of antenna 74 is not axi-symmetric.

Without a secondary launch adapter, the radiation pattern produced by the diagonally-cut antenna may be not linear (e.g. co-axial or parallel to the main cable axis).

The antenna of the present embodiment may be suited to radiate sufficiently to cause localised tissue ablation of targeted tissue volumes. In further embodiments, the antenna may be suited to radiate sufficiently to cause tissue heating without causing ablation. In some embodiments, the antenna may be suited for causing hyperthermia. The antenna performance may vary with the applied excitation frequency at the energy source. The excitation frequency at the energy source may be used to control a depth of ablation penetration by antenna angle. The antenna may form part of a microwave surgical instrument for the precision directional ablation of targeted tissue volumes.

The antenna may be simple and/or easy to manufacture. There may be no need for a choke to tune to a different frequency. Surface currents may be reduced or eliminated. The antenna is directional. The antenna may be considered to have a built-in shield.

The precise ablation that may be provided by the directional antenna may be used to address surgical procedures in difficult and/or critical areas. Difficult areas may include, for example, in which other tissue that must not be treated is within an area that would be irradiated if omnidirectional microwave energy were used to treat the primary target. Controlling a volume of ablation may bring benefits of microwave ablation to new procedures. For example, in superficial and/or dermal applications, 2D areas of ablation with a shallow depth of penetration may be used. As another example, focused linear delivery may be appropriate for atrial fibrillation therapies. Focused linear delivery may be appropriate for treatment close to important physiological structures such as major arteries. The creation of 2D areas with a shallow depth of penetration and/or focused linear delivery may be difficult to achieve with some existing technologies without introducing tool complexity and manufacture cost.

Treatment within the human circulation system where catheter delivery is the preferred route may involve miniaturised dimensions and/or simple compact design features to ensure compatibility. Delivering sufficient microwave power to ablate at a length from the energy source, as may be the case in catheter delivery, may involve highly efficient transmission methods combined with efficient antenna designs.

The present embodiment may provide highly efficient transmission and an efficient antenna design. By contrast, some known devices having features at the distal end of a microwave probe assembly may introduce reflection coefficients and reduce the power delivered to the target body. In such devices, higher power transmission may increase losses inherent in the coaxial line, which may introduce unwanted heating effects along the length of the coaxial line. In such devices, transitions and/or impedance transformers may contain regions of intense energy density resulting in excess heating at those locations.

Coaxial cables may often be chosen to suit applications. The choice of a coaxial cable may be driven by cost and performance. The demands for delivering microwaves down a lumen may mean that the possible diameter of the coaxial cable is limited, and the efficiency of transmission to radiation may be chosen to be high. In the present embodiment, by not introducing reflection components into the delivery as is the case in some known systems, the efficiency may be maximised.

In embodiments in which the antenna device is small, the minimally complex profile of the antenna profile may make the device suitable for placement into confined regions. The antenna may deliver precise, controlled microwave ablation. The small size and minimally complex profile of the antenna may make the antenna suitable for catheter and/or trocar delivery into humans or other animals.

The simple nature of the cut to the coaxial cable may make the ease of manufacture of the antenna of the present embodiment far greater than some other antenna designs. In some other antenna designs, it may be the case that tools, reflectors and/or shields need to be coupled to the transmission cable. In some antenna designs, the cable itself may require specialist manufacturing processes.

In hepatobiliary surgery operations high blood perfusion may be a common issue. Using the directional microwave applicator of FIG. 1, a surface may be ablated pre-incision to reduce operative blood loss. The directional microwave applicator may be used in oncology surgery procedures, for example for pancreatic cancer and brain tumour surgery. The directional microwave applicator may facilitate precise treatment of structures. Further exemplary applications of the directional microwave applicator may comprise treatment for pain management, ablation of sinus tracts and tonsillectomy. Linear ablation may be used for atrial fibrillation maze procedures.

Existing RF techniques may generate smoke, which may impair visibility at the time of sealing. Existing RF techniques may have the potential for nerve damage to be incurred. The microwave technique described above may, in some circumstances, generate substantially no smoke and may, in some circumstances, not result in nerve damage.

In the embodiment described above with reference to FIG. 1, the microwave apparatus is a directional microwave ablation instrument for the targeted ablation of biological tissue. The instrument includes a transmission line having a proximal portion suitable for connection to an electromagnetic energy source and an antenna area having a longitudinal axis and a 2D surface. The antenna area may be described as being coupled to the transmission line.

The antenna of FIG. 1 is formed by planar tangential slicing of the cylindrical coaxial transmission line with no additional components. The angle of slice may dictate an operating frequency of the antenna portion, and/or the match into surrounding media of the antenna portion.

The directional antenna described above has an angled cut made across the entirety of the coaxial cable, e.g. slicing through the inner conductor 1, dielectric 2, outer conductor 3 and jacket 4. The angled cut is substantially planar. The angled cut forms a single elliptical surface radiating directional microwave energy.

In other embodiments, the radiating surface may be not elliptical. The radiating surface may be a portion of an ellipse as is the case in the embodiment of FIGS. 5 and 6. The radiating surface may be of any appropriate shape. In other embodiments, multiple radiating surfaces may be formed. A multi-faceted 2D surface may be formed.

In the embodiments above, the end surface is substantially planar. In other embodiments, the end surface 12 may be curved or may have any other appropriate shape. The end surface 12 may be faceted.

FIG. 10 is a schematic diagram of a coaxial cable having a curved end surface. In the embodiment of FIG. 10, the insulator is flush with the inner and outer conductors. A curvature of the insulator corresponds to a curvature of the outer conductors. Any curvature profile may be used.

In the embodiment of FIG. 1, the antenna is formed from the coaxial cable that is coupled to the microwave generator 20. In other embodiments, the antenna may comprise a separate coaxial component that is coupled to a coaxial cable that is itself coupled to the microwave generator 20. The antenna may be formed from a separate piece of coaxial cable from the coaxial cable that is attached to the microwave generator 20. The antenna may be detachable. The antenna may be disposable.

In the embodiments described above, a single directional applicator comprising a portion of a coaxial cable having an angled cut surface is used to radiate microwave energy into tissue.

In other embodiments, a plurality of directional applicators (for example, a plurality of the directional applicator described above) are placed around a tumour or other target. The plurality of directional applicators may direct energy specifically towards the target from a periphery of the target whilst avoiding radiating into healthy tissue.

Although certain uses for a directional antenna are described above, the directional antenna may be used for any appropriate process. In some embodiments, the directional antenna does not perform ablation. The directional antenna may perform any desired tissue heating process. For example, the directional antenna may provide more mild temperature elevation than may be used for an ablation process. The more mild temperature elevation may be used for hyperthermia. In some circumstances, lower temperatures may be used for surface applications than for penetration applications.

Whether ablation or hyperthermic treatment is performed may be dependent on energy dose. A more dense energy dose may result in heating tissue to a hotter temperature and/or heating tissue more quickly. In some circumstances, a desired result of heating may be cell death. In some circumstances, a desired result of heating may be a call heat reaction, which may not comprise cell death. Parameters (for example, parameters of the antenna and/or of the energy supplied to the antenna) may be selected in order to obtain a desired result of heating.

Embodiments of the directional antenna may be used for any appropriate process involving microwave ablation or heating (for example, hyperthermia) of human or animal tissue. The microwave ablation or heating may be performed on any human or animal subject.

It will be understood that the present invention has been described above purely by way of example, and modifications of detail can be made within the scope of the invention. Each feature disclosed in the description, and (where appropriate) the claims and drawings may be provided independently or in any appropriate combination.

What is claimed is:

1. A microwave system comprising:
a microwave generator; and
a microwave cable apparatus comprising a coaxial cable having a diagonally-angled end surface;
wherein the microwave generator is configured to provide microwave energy to the cable apparatus at a frequency that provides directional radiation of microwave energy having a desired directionality from the diagonally-angled end surface.

2. The system according to claim 1, wherein the system is configured to perform microwave ablation of tissue.

3. The system according to claim 1, wherein the diagonally-angled end surface forms a diagonal angle with a longitudinal axis of the coaxial cable.

4. The system according to claim 1, wherein the coaxial cable comprises an inner conductor and an outer conductor, and wherein one side of the outer conductor is longer than the inner conductor and an opposite side of the outer conductor is shorter than the inner conductor.

5. The system according to claim 4, wherein the end surface comprises an end surface of the outer conductor and an end surface of the inner conductor.

6. The system according to claim 4, wherein the coaxial cable further comprises a dielectric interposed between the inner conductor and the outer conductor, and wherein the end surface comprises an end surface of the dielectric.

7. The system according to claim 1, wherein the end surface is substantially planar.

8. The system according to claim 1, wherein the end surface is substantially elliptical.

9. The system according to claim 1, wherein the end surface is curved and/or faceted.

10. The system according to claim 1, further comprising a controller configured to select the frequency of the microwave energy provided to the cable apparatus and/or a power of the microwave energy provided to the cable apparatus.

11. The system according to claim 9, wherein the frequency and/or power is selected in dependence on at least one of a reflection coefficient of the cable apparatus, a property of tissue to be treated, a volume of tissue to be treated, a type of treatment.

12. The system according to claim 1, wherein the desired directionality comprises at least one of a desired depth of penetration into tissue, a desired radiation pattern, a desired linearity, a desired profile of radiated volume.

13. The system according to claim 1, wherein the coaxial cable is flexible.

14. The system according to claim 1, further comprising a catheter or trocar into which the cable apparatus is insertable.

15. The system according to claim 1, wherein the cable apparatus further comprises a coating that coats at least part of the end surface.

16. The system according to claim 15, wherein the coating is biocompatible.

17. The system according to claim 4, the cable apparatus further comprising a jacket surrounding the outer conductor.

18. The system according to claim 1, wherein the diagonal angle of the diagonally-angled end surface is between 10° to 85°, optionally between 30° and 60°.

19. The system according to claim 1, wherein the diagonal angle of the diagonally-angled end surface is selected in dependence on at least one of: a volume of tissue to be treated, a property of tissue to be treated, a dielectric constant of tissue to be treated, a type of treatment.

20. The system according to claim 1, wherein the frequency is between 900 MHz and 30 GHz, optionally wherein the frequency is about 915 MHz, about 2.45 GHz, about 5.8 GHz, about 8.0 GHz, or about 24.125 GHz.

21. The system according to claim 1, wherein a diameter of the coaxial cable is between 0.1 mm and 25 mm.

22. The system according to claim 1, wherein the end surface is alignable with a tissue feature, so as to radiate directionally into the tissue feature.

23. A method of performing a tissue heating process comprising:
generating microwave energy by a microwave generator;
providing the microwave energy to a microwave cable apparatus comprising a coaxial cable having a diagonally-angled end surface; and
heating tissue by directional radiation of microwave energy having a desired directionality from the diagonally-angled end surface.

24. The method according to claim 23, wherein the tissue heating is so as to perform tissue ablation.

25. The method according to claim 23, wherein the tissue heating is so as to cause tissue hyperthermia.
